SEQUENCE LISTING (1 sequence)

SEQ ID NO: 1 - DNA, 47 bp, Artificial Sequence
  chemically synthesized
acatactggacgtancngnacaagaagantncngcatcatgagagac

(19) United States
(12) Patent Application Publication
(10) Pub. No.: US 2017/0211139 A1
(43) Pub. Date: Jul. 27, 2017

(54) COMPOSITIONS AND METHODS FOR DETECTING RARE SEQUENCE VARIANTS IN NUCLEIC ACID SEQUENCING

(71) Applicant: EXOSOME DIAGNOSTICS, INC., Cambridge, MA (US)

(72) Inventors: Mikkel Noerholm, Gauting (DE); Daniel Enderle, Martinsried (DE)

(21) Appl. No.: 15/325,499

(22) PCT Filed: Jul. 13, 2015
(86) PCT No.: PCT/US15/40160

§ 371 Date: Jan. 11, 2017

Related U.S. Application Data
(60) Provisional application No. 62/023,497, filed on Jul. 11, 2014.

(51) Int. Cl. C12Q 1/68

(52) U.S. Cl. CPC C12Q 1/6869; C12Q 1/6848

(57) ABSTRACT

The present invention relates to compositions that include one or more control molecules known as artificial reference sequences and methods of using these control molecules for estimating rare nucleic acid sequence variants from low copy numbers in ultra-deep sequencing.

### RELATED APPLICATIONS

This application claims priority to and the benefit of U.S. Provisional Application No. 62/023,497, filed Jul. 11, 2014, the contents of which are hereby incorporated by reference in its entirety.

### FIELD OF INVENTION

The present invention relates to compositions that include one or more control molecules known as artificial reference sequences and methods of using these control molecules for estimating rare nucleic acid sequence variants from low copy numbers in ultra-deep sequencing.

### BACKGROUND

The development of next generation nucleic acid sequencing techniques, also known as NGS, provides the capacity to analyze hundreds of billions of base pairs at small fraction of the time and cost of previous sequencing methods. However, it can be difficult using these NGS techniques to detect nucleic acid sequences that appear in low copy numbers in a given biological sample.

Accordingly, there exists a need for controls and methods that allow for estimating the frequencies of these nucleic acid sequences from low copy numbers in a sequencing pipeline.

### SUMMARY OF THE INVENTION

The present invention is directed to compositions and methods for providing an in-process control for nucleic acid sequencing techniques, including, for example, next-generation sequencing (NGS) assays, to detect low-frequency sequence variants. These controls provide a number of technical advantages.

In some embodiments, the compositions and/or methods include an artificial reference sequence (ARS). In some embodiments, the ARS is an oligonucleotide that contains a predetermined number of defined mutations, e.g., at least one defined mutation, at least two or more, at least three or more, at least four or more, at least five or more defined mutations.

The compositions and methods provided herein are particularly useful in NGS assays to detect low-frequency sequence variants in nucleic acids isolated and/or extracted from biological samples.

In some embodiments, the compositions and methods are used to analyze nucleic acids isolated and/or extracted from the microvesicle fraction of a biological sample. Small membrane-bound vesicles shed by cells are described as “microvesicles”. Microvesicles may include exosomes, exosome-like particles, prostasomes, dexosomes, texosomes, ectosomes, oncosomes, apoptotic bodies, retrovirus-like particles, and human endogenous retrovirus (HERV) particles. Studies have shown that microvesicles are shed from many different cell types under both normal and pathological conditions. Importantly, microvesicles have been shown to contain DNA, RNA, and proteins. Recent studies have shown that the analysis of the contents of microvesicles has revealed that biomarkers, or disease-associated genes can be detected, therefore, demonstrating the value of microvesicle analysis for aiding in the diagnosis, prognosis, monitoring, or therapy selection for a disease or other medical disease.

Various nucleic acid sequencing techniques are used to detect and analyze nucleic acids such as cell free DNA and/or RNA extracted from the microvesicle fraction from biological samples. Analysis of nucleic acids such as cell free DNA and/or nucleic acids extracted from microvesicles for diagnostic purposes has wide-ranging implications due to the non-invasive nature in which microvesicles can be easily collected. Use of microvesicle analysis in place of invasive tissue biopsies will positively impact patient welfare, improve the ability to conduct longitudinal disease monitoring, and improve the ability to obtain expression profiles even when tissue cells are not easily accessible (e.g., in ovarian or brain cancer patients).

Thus, the controls and methods provided here are additional tools to ensure the consistency, reliability, and practicality of diagnostic microvesicle analysis for use in the clinical field. In particular, these controls and methods allow reliable estimating of the frequencies of rare variant nucleic acid sequences from low copy numbers in a NGS sequencing pipeline.

In some embodiments, the control molecule is an artificial reference sequence (ARS) comprising the nucleic acid sequence:

(SEQ ID NO: 1)
acatactggacgtaX₁cX₂gX₃acaagaagaX₄tX₅cX₆gcatcatgaga
gac,

where X₁ has the following variability: A=5%, C=5%, G=85%, and T=5%; X₂ has the following variability: A=0%, C=5%, G=0%, and T=95%; X₃ has the following variability: A=5%, C=0%, G=95%, and T=0%; X₄ has the following variability: A=10%, C=0%, G=90% and T=0%; X has the following variability: A=75%, C=0%, G=25%, and T=0%; and X₆ has the following variability: A=50%, C=0%, G=50%, and T=0%.

In some embodiments, this ARS is used as a control in a method of analyzing nucleic acids extracted from a biological sample, In some embodiments, the nucleic acids are extracted from the microvesicle fraction of the biological sample. In some embodiments, the method of analyzing nucleic acids is ultra-deep sequencing. In some embodiments, the ARS is spiked in as a control in ultra-deep sequencing during pre-amplification, library preparation, sequencing, or any combination thereof.

The biological sample is a bodily fluid. The bodily fluids can be fluids isolated from anywhere in the body of the subject, preferably a peripheral location, including but not limited to, for example, blood, plasma, serum, urine, sputum, spinal fluid, cerebrospinal fluid, pleural fluid, nipple aspirates, lymph fluid, fluid of the respiratory, intestinal, an genitourinary tracts, tear fluid, saliva, breast milk, fluid from the lymphatic system, semen, cerebrospinal fluid, intra-organ system fluid, ascitic fluid, tumor cyst fluid, amniotic fluid and combinations thereof For example, the bodily fluid is urine, blood, serum, or cerebrospinal fluid.

In any of the foregoing methods, the nucleic acids are DNA or RNA. Examples of RNA include messenger RNAs, transfer RNAs, ribosomal RNAs, small RNAs (non-protein-coding RNAs, non-messenger RNAs), microRNAs, piRNAs, exRNAs, snRNAs and snoRNAs.

Various aspects and embodiments of the invention will now be described in detail. It will be appreciated that modification of the details may be made without departing from the scope of the invention. Further, unless otherwise required by context, singular terms shall include pluralities and plural terms shall include the singular.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representations as to the contents of these documents are based on the information available to the applicants and do not constitute any admission as to the correctness of the dates or contents of these documents.

### BRIEF DESCRIPTION OF THE DRAWINGS

FIG. 1 is an illustration of an artificial reference sequence (ARS) embodiment, the 128 distinct hexamers created from this ARS, and the relative frequency of each distinct ARS version.

FIG. 2 is a schematic representation of the ultra-deep sequencing of PCR amplicons.

FIG. 3 is a graph depicting the recovery of the expected percentages of each hexamer.

FIG. 4 is a graph depicting that detection rate is largely driven by low copy thinkers.

FIG. 5 is a graph depicting the reproducibility and accuracy from repeated sequencing results.

### DETAILED DESCRIPTION OF THE INVENTION

Biofluids contain nucleic acids, either as cell-free DNA or captured in exosomes and other microvesicles, which are stable sources of genetic material for personalized medicine. Biofluids are easy to access and allow genotyping of solid tumors without requiring tissue. Low numbers of somatic mutations are diluted in a sea of wild-type sequences; targeted ultra-deep sequencing is our method of choice for the detection of rare variants.

The compositions and methods provided herein address the question of how well mutation frequencies can be estimated from low copy numbers in nucleic acid sequencing workflow. In the working examples provided herein, short DNA sequences were synthesized. These short DNA sequences were identical except for 6 positions, where single nucleotide variations of pre-specified frequency were introduced, such that their combination generates 128 distinct sequences with relative frequencies between 26% and 0.0002%. Paired-end sequencing where both forward and reverse read covered the entire 87 nucleotides of the synthetic DNA was performed. Sequences where forward and reverse read did not agree were filtered, to increase the precision of the obtained sequences.

The results demonstrated in the working examples show almost perfect recovery of the expected percentages with a Pearson coefficient of 0.99 between input and observation. The variance in counts of rare sequences follows that of a Poisson distribution. Moreover, at a coverage of 40,000 reads, there is a pickup rate of 100% down to frequencies of 0.004%, corresponding to 1.6 molecules detected in the sample. In conclusion, the limiting factor for estimating the frequency of rare variants is determined by a Poisson distribution at very leer copy numbers, rather than systematic errors due to the experimental procedure.

The invention provides compositions and methods for detecting rare sequences, rare sequence variants, in nucleic acid sequencing techniques. For example, these compositions and methods are useful for detecting rare sequences, those having a low copy number in a biological sample, in targeted ultra-deep sequencing methods.

Using the compositions and methods provided herein, single molecules can be picked up by the ultra-deep sequencing pipeline.

An artificial reference sequence (ARS) is used to control the entire process from pre-amplification, library preparation, and sequencing.

At a coverage of 350 k, the compositions and methods described herein provide a detection rate for hexamers of 100% down to 0.00141%.

The limiting factor for estimating the frequency of rare variants is determined by a Poisson distribution.

The compositions and methods described herein provide excellent reproducibility of the entire pipeline with an coefficient of determination of 0.9975.

The compositions and methods described herein are useful in analyzing sequences derived from biological samples, including cell free DNA and/or nucleic acids extracted from the microvesicle fraction of the biological sample.

All membrane vesicles shed by cells <0.8 μm in diameter are referred to herein collectively as microvesicies. This may include exosomes, exosome-like particles, prostasomes, dexosomes, texosomes, ectosomes, oncosomes, apoptotic bodies, retrovirus-like particles, and human endogenous retrovirus (HERV) particles. Microvesicles from various cell sources have been extensively studied with respect to protein and lipid content.

Microvesicles have been previously shown to be valuable diagnostic and prognostic tools. An initial study demonstrated that glioblastoma-derived microvesicles could be isolated from the serum of glioblastorna patients. Importantly, these microvesicles contain mRNA associated with the tumor cells. The nucleic acids within these microvesicles can be used as valuable biomarkers for tumor diagnosis, characterization and prognosis. For example, the nucleic acids within the microvesicles could be used to monitor tumor progression over time by analyzing if other mutations are acquired over time or over the course of treatment. In addition, levels of disease-associated genes can also be determined and compiled into a genetic expression profile which can be compared to reference profiles to diagnose or prognose a disease or monitor the progression of a disease or therapeutic regimen.

In some embodiments, biological samples are first processed to remove cells and other large contaminants. This first pre-processing step can be accomplished by using a 0.8 μm filter to separate cells and other cell debris from the microvesicles. Optionally, centrifugation (i.e., slow centrifugation) can be used to further separate contaminants from the microvesicles. Control particles can be added to the pre-processed sample at a known quantity. Additional processing is performed to isolate a fraction containing microvesicles and control particles. Suitable additional processing steps include filtration concentrators and differential centrifugation. The fraction containing microvesicles and control particles is washed to remove additional contaminants at least once. The fraction may be washed once, twice, three times, four times, five times using a physiological buffer, such as phosphate. buffered saline. RNase inhibitor was added to the fraction, preferably to the fraction located in the upper chamber of the filter concentrator. Lysis of the microvesicles and control particles can be optionally performed in the upper chamber of the filter concentrator.

The method of isolating microvesicles from a biological sample and extracting nucleic acids from the isolated microvesicles may be achieved by many methods. Some of these methods are described in publications WO 2009/100029 and WO 2011/009104, both of which are hereby incorporated in their entirety. In one embodiment, the method comprises the following steps: removing cells from the bodily either by low speed centrifugation and/or filtration though a 0.8 μm filter; centrifuging the supernatant/filtrate at about 120,000 ×g for about 0.5 hour at about 4° C.; treating the pellet with a pre-lysis solution, e.g., an RNase inhibitor and/or a pH buffered solution and/or a protease enzyme in sufficient quantities; and lysing the pellet for nucleic acid extraction. The lysis of microvesicles in the pellet and extraction of nucleic acids may be achieved with various methods known in the art (e.g., using commercially available kids (e.g., Qiagen) or phenol-chloroform extraction according to standard procedures and techniques known in the art). Control particles can be added, at least, prior to the microvesicle isolation step or prior to the RNA extraction step.

Additional methods of isolating microvesicles from a biological sample are known in the art. For example, a method of differential centrifugation is described by Raposo et al. (Raposo et al., 1996). Methods of anion exchange and/or gel permeation chromatography are described in U.S. Pat. Nos. 6,899,863 and 6,812,023. Methods of sucrose density gradients or organelle electrophoresis are described in U.S. Pat. No. 7,198,923. A method of magnetic activated cell sorting (MACS, Miltenyi) is described in (Taylor and Gercel-Taylor, 2008), A method of nanomembrane ultrafiltration concentrator is described in (Cheruvanky et al., 2007). Preferably, microvesicles can be identified and isolated from bodily fluid of a subject by a newly developed microchip technology that uses a unique microfluidic platform to efficiently and selectively separate tumor derived microvesicles. This technology, as described in a paper by Nagrath et al. (Nagrath et al., 2007), can be adapted to identify and separate microvesicles using similar principles of capture and separation as taught in the paper. Each of the foregoing references is incorporated by reference herein for its teaching of these methods.

In one embodiment, the microvesicles isolated from a bodily fluid are enriched for those originating from a specific cell type, for example, lung, pancreas, stomach, intestine, bladder, kidney, ovary, testis, skin, colorectal, breast, prostate, brain, esophagus, liver, placenta, fetus cells. Because the microvesicles often carry surface molecules such as antigens from their donor cells, surface molecules may be used to identify, isolate and/or enrich for microvesicles from a specific donor cell type (Al-Nedawi et al., 2008; Taylor and Gercel-Taylor, 2008). In this way, microvesicles originating from distinct cell populations can be analyzed for their RNA content. For example, tumor (malignant and nonmalignant) rnicrovesicles carry tumor-associated surface antigens and may be detected, isolated and/or enriched via these specific tumor-associated surface antigens. In one example, the surface antigen is epithelial-cell-adhesion-molecule (EpCAM), which is specific to microvesicles from carcinomas of lung, colorectal, breast, prostate, head and neck, and hepatic origin, but not of hematological cell origin (Balzar et al., 1999; Went et al., 2004). In another example, the surface antigen is CD24, which is a glycoprotein specific to urine microvesicles (Keller et al., 2007). In yet another example, the surface antigen is selected from a group of molecules CD70, carcinoembryonic antigen (CEA), EGFR, EGFRvIII and other variants, Fas ligand, TRAIL, transferrin receptor, p38.5, p97 and HSP72. Additionally, tumor specific microvesicles may be characterized by the lack of surface markers, such as CD80 and CD86.

The isolation of microvesicles from specific cell types can be accomplished, for example, by using antibodies, aptamers, aptamer analogs or molecularly imprinted polymers specific for a desired surface antigen. In one embodiment, the surface antigen is specific for a cancer type. In another embodiment, the surface antigen is specific for a cell type which is not necessarily cancerous. One example of a method of microvesicle separation based on cell surface antigen is provided in U.S. Pat. No. 7,198,923. As described in, e.g., U.S. Pat. Nos. 5,840,867 and 5,582,981, WO2003/050290 and a publication by Johnson et al. (Johnson et al., 2008), aptamers and their analogs specifically bind surface molecules and can be used as a separation tool for retrieving cell type-specific microvesicles. Molecularly imprinted polymers also specifically recognize surface molecules as described in, e.g., U.S. Pat. Nos. 6,525,154, 7,332,553 and 7,384,589 and a publication by Bossi et al. (Bossi et al., 2007) and are a tool for retrieving and isolating cell type-specific microvesicles. Each of the foregoing reference is incorporated herein for its teaching of these methods.

In some embodiments, it may be beneficial or otherwise desirable to amplify the nucleic acid of the microvesicle prior to analyzing it. Methods of nucleic acid amplification are commonly used and generally known in the art, many examples of which are described herein. If desired, the amplification can be performed such that it is quantitative. Quantitative amplification will allow quantitative determination of relative amounts of the various nucleic acids, to generate a genetic or expression profile.

In one embodiment, the nucleic acid extracted from the microvesicles is DNA. In one embodiment, the nucleic acid extracted from the microvesicles is RNA. RNA may include messenger RNAs, transfer RNAs, ribosomal RNAs, small RNAs (non-protein-coding RNAs, non-messenger RNAs), microRNAs, piRNAs, exRNAs, snRNAs and snoRNAs.

In some aspects, the RNA is preferably reverse-transcribed into complementary DNA (cDNA) before further amplification. RNAs are then preferably reverse-transcribed into complementary DNAs before further amplification. Such reverse transcription may be performed alone or in combination with an amplification step. One example of a method combining reverse transcription and amplification steps is reverse transcription polymerase chain reaction (RT-PCR), which may be further modified to be quantitative, e.g., quantitative RT-PCR as described in U.S. Pat. No. 5,639,606, which is incorporated herein by reference for this teaching. The extracted nucleic acids or complementary DNA can be analyzed for diagnostic purposes by nucleic acid amplification.

Nucleic acid amplification methods include, without limitation, polymerase chain reaction (PCR) (U.S. Pat. No. 5,219,727) and its variants such as in situ polymerase chain reaction (U.S. Pat. No. 5,538,871), quantitative polymerase chain reaction (U.S. Pat. No. 5,219,727), nested polymerase chain reaction (U.S. Pat. No. 5,556,773), self-sustained sequence replication and its variants (Guatelli et al., 1990), transcriptional amplification system and its variants (Kwoh et al., 1989), Qb Replicase and its variants (Miele et al., 1983), cold-PCR (Li et al., 2008), BEAMing (Li et al., 2006) or any other nucleic acid amplification methods, followed by the detection of the amplified molecules using techniques well known to those of skill in the art. Especially useful are those detection schemes designed for the detection of nucleic acid molecules if such molecules are present in very low numbers. The foregoing references are incorporated herein for their teachings of these methods. In other embodiment, the step of nucleic acid amplification is not performed. Instead, the extract nucleic acids are analyzed directly (e.g., through next-generation sequencing).

The analysis of nucleic acids present in the isolated particles is quantitative and/or qualitative. For quantitative analysis, the amounts or expression levels, either relative or absolute, of specific nucleic acids of interest within the isolated particles are measured with methods known in the art. For qualitative analysis, the species of specific nucleic acids of interest within the isolated particles, whether wild type or variants, are identified with methods known in the art.

The present invention also includes methods for microvesicle nucleic acid analysis with the presence of control particles for (i) aiding in the diagnosis of a subject, (ii) monitoring the progress or reoccurrence of a disease or other medical condition in a subject, or (iii) aiding in the evaluation of treatment efficacy for a subject undergoing or contemplating treatment for a disease or other medical condition; wherein the presence or absence of one or more biomarkers in the nucleic acid extraction obtained from the method is determined, and the one or more biomarkers are associated with the diagnosis, progress or reoccurrence, or treatment efficacy, respectively, of a disease or other medical condition.

The one or more biomarkers can be one or a collection of genetic aberrations, which is used herein o refer to the nucleic acid amounts as well as nucleic acid variants within the nucleic acid-containing particles. Specifically, genetic aberrations include, without limitation, over-expression of a gene (e.g., an oncogene) or a panel of genes, under-expression of a gene (e.g., a tumor suppressor gene such as p53 or RB) or a panel of genes, alternative production of splice variants of a gene or a panel of genes, gene copy number variants (CNV) (e.g., DNA double minutes) (Hahn, 1993), nucleic acid modifications (e.g., methylation, acetylation and phosphorylations), single nucleotide polymorphisms (SNPs), chromosomal rearrangements (e.g., inversions, deletions and duplications), and mutations (insertions, deletions, duplications, missense, nonsense, synonymous or any other nucleotide changes) of a gene or a panel of genes, which mutations, in many cases, ultimately affect the activity and function of the gene products, lead to alternative transcriptional splice variants and/or changes of gene expression level, or combinations of any of the foregoing.

The determination of such genetic aberrations can be performed by a variety of techniques known to the skilled practitioner. For example, expression levels of nucleic acids, alternative splicing variants, chromosome rearrangement and gene copy numbers can be determined by microarray analysis (see, e.g., U.S. Pat. Nos. 6,913,879, 7,364,848, 7,378,245, 6,893,837 and 6,004,755) and quantitative PCR. Particularly, copy number changes may be detected with the Illumina Infinium II whole genome genotyping assay or Agilent Human Genome CGH Microarray (Steemers et al., 2006). Nucleic acid modifications can be assayed by methods described in, e.g., U.S. Pat. No. 7,186,512 and patent publication WO2003/023065. Particularly, methylation profiles may be determined by Illumina DNA Methylation OMA003 Cancer Panel. SNPs and mutations can be detected by hybridization with allele-specific probes, enzymatic mutation detection, chemical cleavage of mismatched heteroduplex (Cotton et al., 1988), ribonuclease cleavage of mismatched bases (Myers et al., 1985), mass spectrometry (U.S. Pat. Nos. 6,994,960, 7,074,563, and 7,198,893), nucleic acid sequencing, single strand conformation polymorphism (SSCP) (Orita et al., 1989), denaturing gradient gel electrophoresis (DGGE)(Fischer and Lerman, 1979a; Fischer and Lerman, 1979b), temperature gradient gel electrophoresis (TGGE) (Fischer and Lerman, 1979a; Fischer and Lerman, 1979b), restriction fragment length polymorphisms (RFLP) (Kan and Dozy, 1978a; Kan and Dozy, 1978b), oligonucleotide ligation assay (OLA), allele-specific PCR (ASPCR) (U.S. Pat. No. 5,639,611), ligation chain reaction (LCR) and its variants (Abravaya et al., 1995; Landegren et al., 1988; Nakazawa et al., 1994), flow-cytometric heteroduplex analysis (WO/2006/113590) and combinations/modifications thereof. Notably, gene expression levels may be determined by the serial analysis of gene expression (SAGE) technique (Velculescu et al., 1995). In general, the methods for analyzing genetic aberrations are reported in numerous publications, not limited to those cited herein, and are available to skilled practitioners. The appropriate method of analysis will depend upon the specific goals of the analysis, the condition/history of the patient, and the specific cancer(s), diseases or other medical conditions to be detected, monitored or treated. The forgoing references are incorporated herein for their teaching of these methods.

Many biomarkers may be associated with the presence or absence of a disease or other medical condition in a subject. Therefore, detection of the presence or absence of such biomarkers in a nucleic acid extraction from isolated particles, according to the methods disclosed herein, may aid diagnosis of the disease or other medical condition in the subject. For example, as described in WO 2009/100029, detection of the presence or absence of the EGFRvIII mutation in nucleic acids extracted from microvesicles isolated from a patient serum sample may aid in the diagnosis and/or monitoring of glioblastoma in the patient. This is so because the expression of the EGFRvIII mutation is specific to some tumors and defines a clinically distinct subtype of glioma. (Pelloski et al., 2007). For another example, as described in WO 2009/100029, detection of the presence or absence of the TMPRSS2-ERG fusion gene and/or PCA-3 in nucleic acids extracted from microvesicles isolated from a patient urine sample may aid in the diagnosis of prostate cancer in the patient. For another example, detection of presence or absence of the combination of ERG and AMACR in a bodily fluid may aid in the diagnosis of cancer in a patient.

Further, many biomarkers may help disease or medical status monitoring in a subject. Therefore, the detection of the presence or absence of such biomarkers in a nucleic acid extraction from isolated particles, according to the methods disclosed herein, may aid in monitoring the progress or reoccurrence of a disease or other medical condition in a subject. For example, as described in WO 2009/100029, the determination of matrix metalloproteinase (MMP) levels in nucleic acids extracted from microvesicles isolated from an organ transplantation patient may help to monitor the post-transplantation condition, as a significant increase in the expression level of MMP-2 after kidney transplantation may indicate the onset and/or deterioration of post-transplantation complications. Similarly, a significantly elevated level of MMP-9 after lung transplantation, suggests the onset and/or deterioration of bronchiolitis obliterans syndrome.

Many biomarkers have also been found to influence the effectiveness of treatment in a particular patient. Therefore, the detection of the presence or absence of such biomarkers in a nucleic acid extraction from isolated particles, according to the methods disclosed herein, may aid in evaluating the efficacy of a given treatment in a given patient. For example, as disclosed in Table 1 in the publication by Furnari et al. (Furnari et al., 2007), biomarkers, e.g., mutations in a variety of genes, affect the effectiveness of specific medicines used in chemotherapy for treating brain tumors. The identification of these biomarkers in nucleic acids extracted from isolated particles from a biological sample from a patient may guide the selection of treatment for the patient.

In certain embodiments of the foregoing aspects of the invention, the disease or other medical condition is a neoplastic disease or condition (e.g., cancer or cell proliferative disorder), a metabolic disease or condition (e.g., diabetes, inflammation, perinatal conditions or a disease or condition associated with iron metabolism), a neurological disease or condition, an immune disorder or condition, a post transplantation condition, a fetal condition, or a pathogenic infection or disease or condition associated with an infection.

As used herein, the term “biological sample” refers to a sample that contains biological materials such as a DNA, a RNA and/or a protein. In some embodiments, the biological sample may suitably comprise a bodily fluid from a subject. The bodily fluids can be fluids isolated from anywhere in the body of the subject, preferably a peripheral location, including but not limited to, for example, blood, plasma, serum, urine, sputum, spinal fluid, cerebrospinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, tumor cyst fluid, amniotic fluid and combinations thereof. In some embodiments, the preferred body fluid for use as the biological sample is urine. In other embodiments, the preferred body fluid is serum. In still other embodiments, the preferred body fluid is cerebrospinal fluid.

Suitably a biological sample volume of about 0.1 ml to about 30 ml fluid may be used. The volume of fluid may depend on a few factors, e.g., the type of fluid used. For example, the volume of serum samples may be about 0.1 ml to about 2 ml, preferably about 1 ml. The volume of urine samples may be about 10 ml to about 30 ml, preferably about 20 ml.

The term “subject” is intended to include all animals shown to or expected to have nucleic acid-containing particles. In particular embodiments, the subject is a mammal, a human or nonhuman primate, a dog, a cat, a horse, a cow, other farm animals, or a rodent (e.g. mice, rats, guinea pig. etc.). A human subject may be a normal human being without observable abnormalities, e.g., a disease. A human subject may be a human being with observable abnormalities, e.g., a disease. The observable abnormalities may be observed by the human being himself, or by a medical professional. The term “subject”, “patient”, and “individual” are used interchangeably herein.

It should be understood that this invention is not limited to the particular methodologies, protocols and reagents, described herein, which may vary. The terminology used herein is for the purpose of describing particular embodiments only, and is not intended to limit the scope of the present invention, which is defined solely by the claims.

Examples of the disclosed subject matter are set forth below. Other features, objects, and advantages of the disclosed subject matter will be apparent from the detailed description, figures, examples and claims. Methods and materials substantially similar or equivalent to those described herein can be used in the practice or testing of the presently disclosed subject matter.

### EXAMPLES

### Example 1

### Computational and Experimental Approaches to the Limit of Detection for Rare Sequence Variants in Targeted Ultra-Deep Sequencing

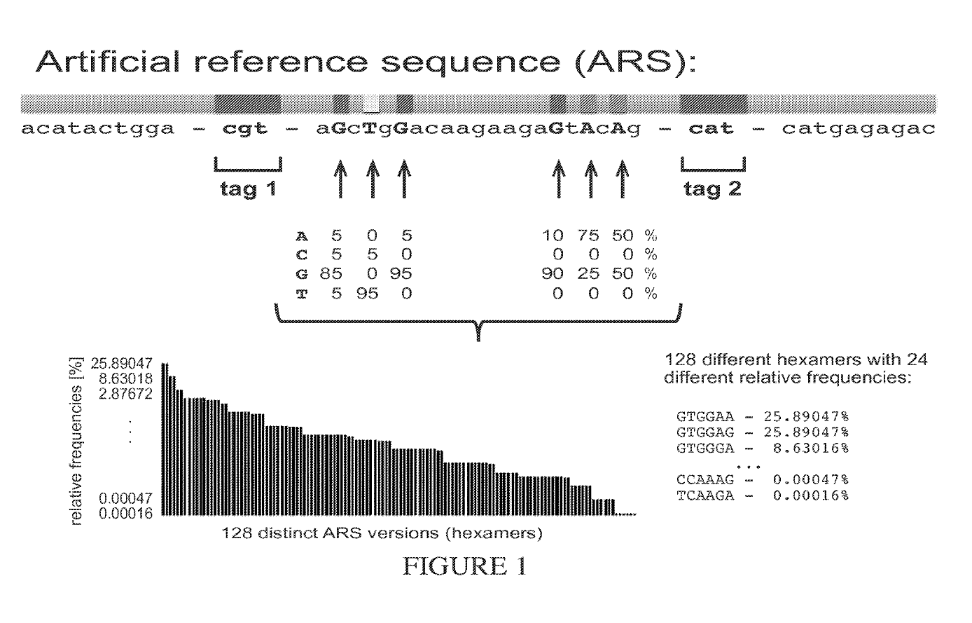

Preparation of an artificial reference sequence: Short DNA sequences were generated to produce an artificial reference sequence (ARS). In particular, these short DNA sequences were identical sequences except for 6 positions, where we introduced single nucleotide variations of pre-specified frequency, such that their combination generates 128 distinct sequences with relative frequencies between 26% and 0.0002%. The sequences are shown in FIG. 1.

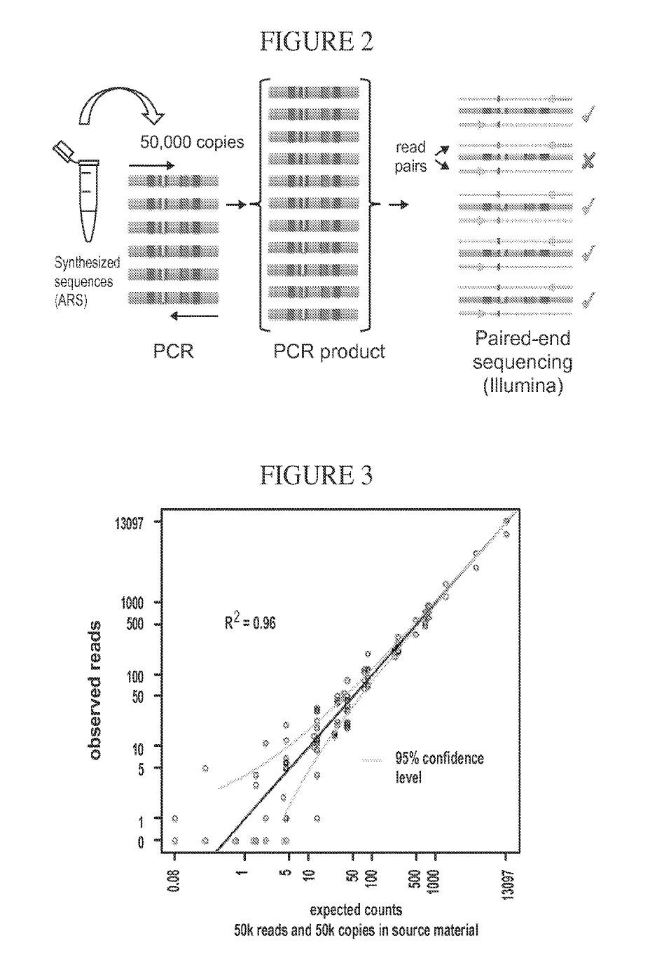

Ultra-deep sequencing of PCR amplicons: Paired-end sequencing was performed where both forward and reverse read covered the entire 87 nucleotides of the synthetic DNA generated. The sequences where forward and reverse read did not agree were filtered to increase the precision of the obtained sequences. The entire procedure was repeated 11 times. An overview of this sequencing is shown in FIG. 2.

Recovery of expected percentages: The scatter plot in FIG. 3 contrasts the number of reads that were expected to be found based on the theoretical percentages of hexamers and the number of reads that were actually found. Black dots represent hexamers that were found and red dots represent hexamers that were not among the ˜50k paired reads. The number of ARS molecules put into the amplification PCR was also around 50k.

The expected number of reads for each hexamer was almost perfectly recovered with an coefficient of determination of 0.956.

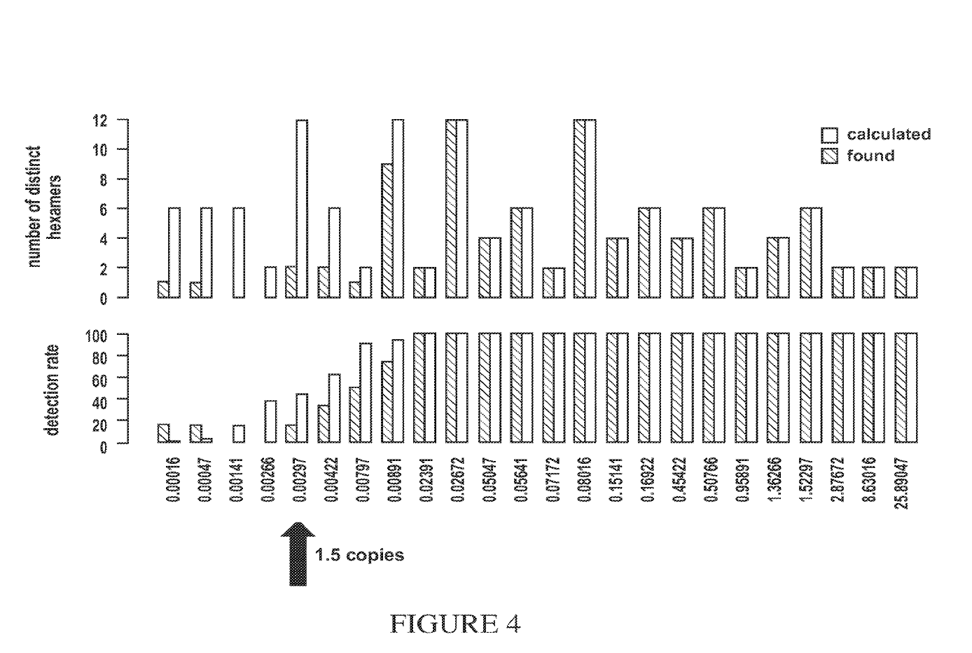

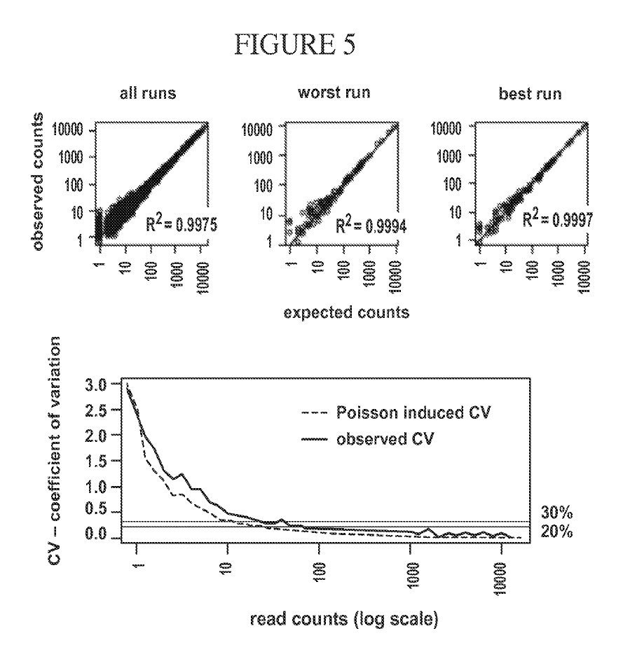

Detection rate: FIG. 4 depicts the detection rate. The detection rate was mostly driven by the low copy numbers. There existed 12 hexamers with an expected frequency of 0.00297% which corresponds to 1.5 copies in the starting material. Given a Poisson distribution, the likelihood of picking up such a low copy number is 44%. In these studies, 2 out of 12 hexamers were found.

Reproducibility and accuracy from repeated sequencing results: In these studies, the ARS composition was measured 11 times with a coverage between 12,268 and 50,586. The mean observed frequency for each hexamer was calculated. The measured frequency was compared with the mean frequency.

The lower limit for accurately estimating the number of very rare variants was determined by the Poisson distribution rather than the experimental procedure. The observed accuracy as defined by the coefficient of variation was close to these theoretical considerations.

While the present invention has been disclosed with reference to certain embodiments, numerous modifications, alterations, and changes to the described embodiments are possible without departing from the full scope of the invention, as described in the appended specification and claims.

1. An artificial reference sequence (ARS) comprising the nucleic acid sequence:
acatactggacgtaX₁cX₂gX₃acaagaagaX₄tX₅cX₆gcatcatgaga
gac
where X₁ has the following variability: A=5%, C=5%, G=85%, and T=5%; X₂ has the following variability: A=0%, C=5%, G=0%, and T=95%; X₃ has the following variability: A=5%, C=0%, G=95%, and T=0%; X₄ has the following variability: A=10%, C=0%, G=90%, and T=0%; X₅ has the following variability: A=75%, C=0%, G=25%, and T=0%; and X₆ has the following variability: A=50%, C=0%, G=50%, and T=0%.

2. Use of the ARS of claim 1 as a control in a method of analyzing nucleic acids extracted from a biological sample.

3. The use of claim 2, wherein the nucleic acids are extracted from the microvesicle fraction of the biological sample.

4. The use of claim 2, wherein the method of analyzing nucleic acids is ultra-deep sequencing.

5. The use of claim 4, wherein the ARS is spiked in as a control in ultra-deep sequencing during pre-amplification, library preparation, sequencing, or any combination thereof.

6. A method of using defined mixtures of nucleotides in a step of oligonucleotide synthesis to create a mixture of oligonucleotides with defined, combinatorial variants of a given sequence, wherein the oligonucleotide mixture is used to assess the ability of a molecular assay to detect DNA variants with a large number frequencies and abundances.

7. The method of claim 6, wherein the oligonucleotide mixture is used as a reference standard, a standard curve to determine absolute copy numbers, an in-process quality control for molecular assays, or any combination thereof.

8. The method of claim 6, wherein the oligonucleotide mixture is spiked into samples at the beginning of, in between steps of a nucleic acid extraction, or both at the beginning of and in between steps of a nucleic acid extraction.

9. The method of claim 6, wherein the oligonucleotide mixture is used as a control in a method of analyzing nucleic acids extracted from a biological sample.

10. The method of claim 6, wherein the method of analyzing nucleic acids is ultra-deep sequencing.

11. The method of claim 6, wherein the oligonucleotide mixture is spiked in as a control in ultra-deep sequencing during pre-amplification, library preparation, sequencing, or any combination thereof.
